(19) United States
(12) Patent Application Publication
(10) Pub. No.: US 2021/0177815 A1
(43) Pub. Date: Jun. 17, 2021

(54) COLONOSCOPY PREPARATION ENHANCER

(71) Applicant: BioGastrex LLC, Tuscaloosa, AL (US)

(72) Inventor: Adisesha Reddy, Tuscaloosa, AL (US)

(21) Appl. No.: 16/716,781

(22) Filed: Dec. 17, 2019

(51) Int. Cl. A61K 31/4402; A61K 9/00; A61K 31/80; A61K 9/20

(52) U.S. Cl. CPC A61K 31/4402; A61K 9/20; A61K 31/80; A61K 9/0095

(57) ABSTRACT

The disclosure provides for a method of treatment, composition of matter and kit to improve the cleansing of the bowel prior to a colonoscopy.

### BACKGROUND

The disclosure provides for a method of treatment, composition of matter and kit to improve the cleansing of the bowel prior to a colonoscopy. Approximately 25% of colonoscopies end up with poor cleansing of the bowel resulting in a less than desirable colonoscopy. The present invention is designed to improve bowel cleansing to enable a superior colonoscopy when compared to the prior art.

### BRIEF SUMMARY

The disclosure provides for a method of treatment, composition of matter and kit to improve the cleansing of the bowel prior to a colonoscopy which is to be combined with traditional prescribed preparations prescribed by the patient's doctor.

### BRIEF DESCRIPTION OF SEVERAL VIEWS OF THE DRAWINGS

The invention will be described in conjunction with the following drawings in which like reference numerals designate like elements and wherein:

FIG. 1 is a diagrammatic representation of a color chart showing various colorations of various stages of stool removal preparation samples contained within a container to enable a patient to determine when their colon preparation is sufficiently complete to enable an appropriate colonoscopy to be performed. One color chart suitable for use is the one described in an abstract entitled “A NOVEL TOOL. ‘COLONOSCOPY PREPARATION CARD USING PATIENT REPORTED STOOL COLOR TO IMPROVE SUCCESS OF DIRECT ACCESS ENDOSCOPY, by Ekta Gupta et al., Gastroenterology 154(6):S-891 May 2018, the entire disclosure of which is incorporated by reference herein.

### DETAILED DESCRIPTION

There are multiple brand names of various colonoscopy preparation methods/compositions sold in the United States. One of them is sold under the brand name SUPREP®. SUPREP® Bowel Prep Kit (sodium sulfate, potassium sulfate and magnesium sulfate) Oral Solution is an osmotic laxative indicated for cleansing of the colon as a preparation for colonoscopy in adults. Most common adverse reactions (>2%) are overall discomfort, abdominal distention, abdominal pain, nausea, vomiting and headache.

Another preparation is sold as MoviPrep and contains PEG-3350, sodium sulfate, sodium chloride, potassium chloride, sodium ascorbate and ascorbic acid for oral solution. Each MoviPrep kit contains a disposable container, 2 pouches labeled A, and 2 pouches labeled B. The patient must complete the entire prep to ensure the most effective cleansing as follows. Step 1: Mix the first dose. At the time instructed by the patient's doctor, Empty 1 pouch A and 1 pouch B into the disposable container. Add lukewarm drinking water to the top line of the container. Mix to dissolve. If preferred, mix the solution ahead of time and refrigerate prior to drinking. The reconstituted solution should be used within 24 hours. Step 2: Drink the first dose. The MoviPrep container is divided by 4 marks. Every 15 minutes, drink the solution down to the next mark (approximately 8 oz.), until the full liter is consumed. The patient is to drink 16 oz of the clear liquid of their choice. This is a necessary step to ensure adequate hydration and an effective prep. Step 3: Mix the second dose. At the time instructed by the patient's doctor, empty 1 pouch A and 1 pouch B into the disposable container. Add lukewarm drinking water to the top line of the container. Mix to dissolve. If preferred, mix the solution ahead of time and refrigerate prior to drinking. The reconstituted solution should be used within 24 hours. Step 4: Drink the second dose. The MoviPrep container is divided by 4 marks. Every 15 minutes, drink the solution down to the next mark (approximately 8 oz.), until the full liter is consumed. Drink 16 oz of the clear liquid of the patient's choice. This is a necessary step to ensure adequate hydration and an effective prep. Acceptable clear liquids include, but are not limited to water, clear fruit juices without pulp including apple, white grape, or white cranberry, strained limeade or lemonade, coffee or tea (without any dairy or non-dairy creamer), clear broth, clear soda, gelatin (without added fruit or topping), popsicles (without pieces of fruit or fruit pulp). Important note: Avoid any red or purple liquid, such as cranberry juice or grape juice, as the coloring can interfere with the results of your exam. Also avoid milk, dairy products, and nondairy coffee creamer. The patient is to drink clear liquids before, during, and after taking MoviPrep to help prevent fluid loss (dehydration). It is important for the patient to drink the additional prescribed amount of clear liquids listed in the Instructions for Use.

Plenvue has the same ingredients as Moviprep. There are some other brands that are being marketed. The colonoscopy prep enhancer of the present invention works with any other brands or generic version of these preps.

The original prep/brand prescribed by the patient's doctor should list the amount of water to be used with the traditional preparation compositions.

According to the present invention, on the evening prior to taking the colonoscopy, the patient takes 250 mg simethicone powder with at least 1-2 glasses of water or clear liquid and about 15-20 mg of biscodyl. This needs to be taken along with the first dose of the prescribed split preparation solution.

The patient is to take another dose of 250 mg simethicone powder with at least 1-2 glasses of water or clear liquid along with the second dose of the prescribed split preparation solution.

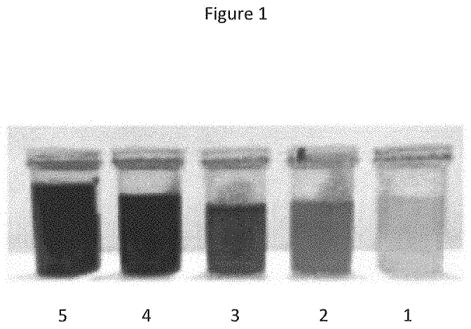

The patient is to check the color of their stool in liquid in their first bowel movement after taking the second dose of the prescribed split preparation by referring to the chart provided in FIG. 1. If the color of the patient's stool in liquid matches the images shown in bottles #3, #4, or #5, then the patient is to take 17 gms of magnesium citrate powder and a second dose of about 15-20 mg biscodyl by mouth with a glass of water or clear fluid, but no red colored fluids should be used. The magnesium citrate needs to be dissolved in water before being ingested by the patient. If the color of their stool matches #1, or #2 of FIG. 1, no further prep will be needed.

The above regimen of the present invention is in addition to the split conventional colonoscopy prep to be prescribed by their gastroenterologist/endoscopist. This is a colon prep enhancer to be used in conjunction with conventional prep solutions.

The scheduling of a colonoscopy time can vary according to the prescribing physician. The regimen of the present invention would not restrict the time the colonoscopy could occur. If the patient has to take Magnesium Citrate, it should be finished 4 hours before the start time of colonoscopy. The bisacodyl will typically be in the form of a tablet made using conventional excipients, however any delivery method would be sufficient, capsule, solution, etc. Simethicone will preferably be in the form of a powder and the magnesium citrate will also be in the form of powder although other conventional delivery forms can be used as desired, e.g., capsule, tablet, solution, etc.

While the invention has been described in detail and with reference to specific examples thereof, it will be apparent to one skilled in the art that various changes and modifications can be made therein without departing from the spirit and scope thereof.

What is claimed is:

1. A method of use of a colonoscopy preparation for a patient undergoing a colonoscopy to assist in removing stool from a patient's colon, the method comprising the following steps:
a) on an evening before the day of the colonoscopy (beginning at about 6 pm-8 pm), the patient takes about 250 mg of simethicone with at least 1-2 glasses or containers of water or clear liquid along with a first dose of a prescribed split preparation solution and about 15-20 mg bisacodyl by mouth;
b) approximately two to six hours after step (a), the patient then takes a dose of about 250 mg of simethicone with at least 1-2 glasses of water or clear liquid along with a second dose of the prescribed split preparation solution; and
c) after a patient's first bowel movement after taking the second dose of the prescribed split preparation solution in step (b), the patient compares the color of their stool in liquid by referring to a color chart as shown in FIG. 1 and if the patient determines that the patient's stool color matches the colors of bottles #1, or #2 of FIG. 1, no further preparation will be needed; however, if the color of the patient's stool in liquid, matches any of the color images shown in bottles #3, #4, or #5 of FIG. 1, then the patient takes about 17 grams of magnesium citrate and optionally takes about 15-20 mg of bisacodyl at least 4 hours prior to a colonoscopy by mouth after first dissolving the magnesium citrate in a container of water or clear fluid.

2. The method of claim 1 wherein the bisacodyl is in tablet form.

3. The method of claim 1 wherein the simethicone is in powder form.

4. The method of claim 1 wherein the bisacodyl is in tablet form and the simethicone is in powder form.

5. The method of claim 1 wherein the magnesium citrate is in powder form.

6. A kit for cleansing a patient's bowel to remove stool from the patient's bowel comprising one or more doses of about 15-20 mg bisacodyl, 250 mg simethicone and optionally an additional 250 mg dose of simethicone and a chart provided in FIG. 1 to compare the color of a patient's stool in liquid to a reference standard to determine if colonoscopy preparation is complete.

7. The method of claim 6 wherein the bisacodyl is in tablet form.

8. The method of claim 6 wherein the simethicone is in powder form.

9. The method of claim 6 wherein the bisacodyl is in tablet form and the simethicone is in powder form.

10. The method of claim 6 wherein the magnesium citrate is in powder form.
